(19) United States
(12) Patent Application Publication
(10) Pub. No.: US 2005/0039220 A1
(43) Pub. Date: Feb. 17, 2005

(54) Imageable animal model of SARS infection

(76) Inventors: Meng Yang, San Diego, CA (US); Mingxu Xu, La Jolla, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/856,529

(22) Filed: May 27, 2004

Related U.S. Application Data
(60) Provisional application No. 60/473,691, filed on May 27, 2003.

(51) Int. Cl. C12Q 1/70; A01K 67/00; C07H 21/04; C07K 14/165

(52) U.S. Cl. 800/8; 435/5; 435/69.3; 435/320.1; 435/325; 530/350; 435/235.1; 435/456; 536/23.72

(57) ABSTRACT

Imaged animal models for coronavirus infection are described.

### RELATED APPLICATION

This application claims the benefit of priority under 35 U.S.C § 119(e) from U.S. Provisional Patent Application No. 60/473,691, filed May 27, 2003, which is hereby incorporated by reference in its entirety.

### TECHNICAL FIELD

The invention relates to a model for coronavirus infection. More particularly, it concerns animals infected with coronavirus that has been labeled with fluorescent protein.

### BACKGROUND ART

Recently, a worldwide outbreak of Severe Acute Respiratory Syndrome (SARS) has caused a substantial number of deaths, disrupted travel plans, and placed thousands of people under quarantine. In fairly short order, using clinical specimens from patients in six countries, it was established that the infection is caused by a coronavirus. See, for example, Ksiazek, T. G., et al., New England J. Med. (2003) 348:1947-1958.

The members of the coronavirus family contain positive-sense RNA genomes of about 30 kb that cause respiratory or intestinal infections in a number of different species. See, for example, de Haan, C. A. M., et al., Virology (2002) 296:177-189. Based on antigenic and genetic criteria, they have been divided into three groups. The common feature of coronaviruses are essential genes encoding replication and structural functions. Interspersed among these genes are group-specific open reading frames (ORFs) that are homologous within each group but that differ among the groups.

The predominant essential gene (ORF) occupies about two-thirds of the genome and is located at the 5′ end of the genome. This gene is a replicase gene that encodes two large precursors, which are cleaved into products for RNA replication and transcription. The other common essential genes code for the four basic structural proteins N, M, E, and S. The nucleocapsid (N) protein packages the viral RNA, forming the core of the virion. This nucleocapsid core structure is surrounded by a lipid envelope in which the membrane (M) protein is most abundant. The small envelope (E) protein and the spike (S) protein are associated with the M protein. The S protein forms the viral peplomers that are involved in virus-cell and cell-cell fusion. These genes are located in the 3′ third of the viral genome. The identities and the locations of the group-specific genes vary, and all their functions have not yet been established. Group 2 viruses, to which mouse hepatitis virus (MHV) belongs, have two group-specific genes, gene 2a and a hemagglutinin-esterase (HE) ORF between ORF 1b and the S gene. Two additional group 2-specific genes, genes 4 and 5a, reside between the S and E genes.

MHV has a single-stranded, positive-sense RNA genome of approximately 31 kb. See, Kim, K. H., et al., J. Virol. (1995) 69:2313-2321. The 5′ end of the MHV genomic RNA contains a 72- to 77-nucleotide-long leader sequence. Downstream of the leader sequence are the MHV-specific genes, each of which is separated by a special short stretch of intergenic sequence. MHV infected cells produce seven major species of virus-specific subgenomic mRNAs. The coronavirus mRNAs are structurally polycistronic, yet produce monocistronic proteins. The coronavirus mRNAs share 3′ ends in a nested-set structure wherein each mRNA is progressively one gene longer than its 3′-neighboring gene, and only the 5′-most gene of each mRNA is translated. These subgenomic mRNAs are named according to their decreasing order of size from 1 to 7. The mRNA sequences are fused with leader sequence at their 5′ ends.

Serial undiluted passage of MHV strain JHM in DBT cells results in generation of defective interfering (DI) RNAs that can be classified into two types. One requires helper virus infection for replication. The other DI type includes DIssA, which is nearly genomic in size, replicates by itself in the absence of helper virus infection, and is packaged into MHV particles Almost all MHV mRNA synthesis is strongly inhibited in DIssA-replicating cells, whereas synthesis of mRNA 7 and its product, N protein, are not inhibited. RNase T1 oligonucleotide fingerprinting analysis of DIssA demonstrate that gene 1 and gene 7 of DIssA are essentially intact, whereas multiple deletions are present from genes 2 to 6. mRNA 7 is synthesized from DIssA template RNA but not from helper virus template RNA, and the gene 1 products and N protein are sufficient for the MHV RNA synthesis.

Thus, it will be sufficient to monitor replication if the DI type DIssA can be labeled. This is the approach illustrated in the present invention.

Fluorescent proteins have been used as fluorescent labels for a number of years. The originally isolated protein emitted green wavelengths and came to be called green fluorescent protein (GFP). Because of this, green fluorescent protein became a generic label for such fluorescent proteins in general, although proteins of various colors including red fluorescent protein (RFP), blue fluorescent protein (BFP) and yellow fluorescent protein (YFP) among others have been prepared. The nature of these proteins is discussed in, for example, U.S. Pat. Nos. 6,232,523; 6,235,967; 6,235,968; and 6,251,384 all incorporated herein by reference. These patents describe the use of fluorescent proteins of various colors to monitor tumor growth and metastasis in transgenic rodents which are convenient tumor models. In addition, these fluorescent proteins have been used to monitor expression mediated by promoters in U.S. application Ser. No. 09/812,710; to monitor infection by bacteria in U.S. Ser. No. 10/192,740 and to monitor cell sorting in U.S. provisional application 60/425,776. The use of fluorescent proteins of different colors to label the nucleus and cytoplasm of cells is disclosed in U.S. provisional applications 60/404,005 and 60/427,604 and mice which are labeled in all tissues, and thus have a consistent fluorescence of the same color are described in U.S. provisional application 60/445,583. All of these documents are incorporated herein by reference.

### DISCLOSURE OF THE INVENTION

The invention provides an animal model wherein fluorescent labeled coronavirus are used to infect susceptible animal subjects, preferably rodents or rabbits, wherein the progress of infection—i.e., the replication of the coronavirus can be followed by monitoring the fluorescence. In a preferred embodiment, the animal is a transgenic animal which comprises tissues that fluoresce in a first color against which the fluorescence of the replicating coronavirus can be readily visualized. The model can be used to determine the effectiveness of vaccines and drugs by viewing, directly, the progress of infection with and without treatment or vaccination. The invention is illustrated below using the DIssA specific sequence from MHV as a model.

Thus, in one aspect, the invention is directed to a coronavirus labeled with a fluorescent protein such as GFP or RFP. In another aspect, the invention is directed to an animal infected with the labeled virus. In still another aspect, the invention is directed to methods to monitor the progress of infection, to evaluate the effectiveness of antiviral drugs, and to evaluate the effects of the vaccines using the animal models of the invention.

### MODES OF CARRYING OUT THE INVENTION

The tools useful in the present invention are described in the U.S. patents and patent applications incorporated by reference above. Whole body imaging, the nature of fluorescent proteins useful in the invention, and methods to label entire animals have been described in these documents.

The disclosed method applicable to coronavirus of the various groups in the coronavirus family. Although in the illustrative example the virus is labeled with RFP and is viewed against a background of a nude mouse expressing GFP in all its tissues, neither the choice of these particular colors nor the use of a labeled animal as a subject is required.

Recombinant Coronavirus

The disclosed invention uses recombinant coronaviruses that are engineered to express a marker, such as a fluorescent protein. By infecting a model organism with the described recombinant coronavirus, one of ordinary skill in the art can use the recombinant virus to study the progression of viral replication in the host animal. Furthermore, the recombinant coronavirus model system has utility as an assay for identifying antiviral agents that slow or inhibit coronavirus replication.

Work by Cornelis, et al. has demonstrated that coronaviruses can by recombinantly engineered to express foreign genes without severe effects on viral replication. Cornelis, et al., J. Virol. (2003) 77(21):11312-11323, which is hereby incorporated by reference in its entirety. The results of this study suggest that position of the foreign gene within the viral genome may impact the viral replication of the recombinant virus vector. Specifically, Cornelis and coworkers observed that expression levels of the foreign gene increased when the foreign gene was inserted closer to the 3′ end of the viral genome. As such, in preferred embodiments of the invention, placement of the fluorescent protein coding sequence occurs toward the 3′ end of the viral genome.

Cornelis and coworkers utilized a murine coronavirus model for their study. The sequence of this virus is well known in the art. At least one variant of the human SARS virus has been sequenced. Marra, M. A., et al., “The Genome sequence of the SARS-associated coronavirus” Science 300 (5624), 1399-1404 (2003). This sequence is publicly available as Accession: NC 004718. The genomic sequence of this SARS variant is provided herein as SEQ ID NO: XX of Table I.

TABLE I
(SEQ ID NO: XX)

| | |
|---|---|
| 1 | atattaggtt tttacctacc caggaaaagc caaccaacct |
| | cgatctcttg tagatctgtt |
| 61 | ctctaaacga actttaaaat ctgtgtagct gtcgctcggc |
| | tgcatgccta gtgcacctac |
| 121 | gcagtataaa caataataaa ttttactgtc gttgacaaga |
| | aacgagtaac tcgtccctct |
| 181 | tctgcagact gcttacggtt tcgtccgtgt tgcagtcgat |
| | catcagcata cctaggtttc |
| 241 | gtccgggtgt gaccgaaagg taagatggag agccttgttc |
| | ttggtgtcaa cgagaaaaca |
| 301 | cacgtccaac tcagtttgcc tgtccttcag gttagagacg |
| | tgctagtgcg tggcttcggg |
| 361 | gactctgtgg aagaggccct atcggaggca cgtgaacacc |
| | tcaaaaatgg cacttgtggt |
| 421 | ctagtagagc tggaaaaagg cgtactgccc cagcttgaac |
| | agccctatgt gttcattaaa |
| 481 | cgttctgatg ccttaagcac caatcacggc cacaaggtcg |
| | ttgagctggt tgcagaaatg |
| 541 | gacggcattc agtacggtcg tagcggtata acactgggag |
| | tactcgtgcc acatgtgggc |
| 601 | gaaaccccaa ttgcataccg caatgttctt cttcgtaaga |
| | acggtaataa gggagccggt |
| 661 | ggtcatagct atggcatcga tctaaagtct tatgacttag |
| | gtgacgagct tggcactgat |
| 721 | cccattgaag attatgaaca aaactggaac actaagcatg |
| | gcagtggtgc actccgtgaa |
| 781 | ctcactcgtg agctcaatgg aggtgcagtc actcgctatg |
| | tcgacaacaa tttctgtggc |
| 841 | ccagatgggt accctcttga ttgcatcaaa gattttctcg |
| | cacgcgcggg caagtcaatg |
| 901 | tgcactcttt ccgaacaact tgattacatc gagtcgaaga |
| | gaggtgtcta ctgctgccgt |
| 961 | gaccatgagc atgaaattgc ctggttcact gagcgctctg |
| | ataagagcta cgagcaccag |
| 1021 | acacccttcg aaattaagag tgccaagaaa tttgacactt |
| | tcaaagggga atgcccaaag |
| 1081 | tttgtgtttc ctcttaactc aaaagtcaaa gtcattcaac |
| | cacgtgttga aaagaaaaag |
| 1141 | actgagggtt tcatggggcg tatacgctct gtgtaccctg |
| | ttgcatctcc acaggagtgt |
| 1201 | aacaatatgc acttgtctac cttgatgaaa tgtaatcatt |
| | gcgatgaagt ttcatggcag |
| 1261 | acgtgcgact ttctgaaagc cacttgtgaa cattgtggca |
| | ctgaaaattt agttattgaa |
| 1321 | ggacctacta catgtgggta cctacctact aatgctgtag |
| | tgaaaatgcc atgtcctgcc |
| 1381 | tgtcaagacc cagagattgg acctgagcat agtgttgcag |
| | attatcacaa ccactcaaac |
| 1441 | attgaaactc gactccgcaa gggaggtagg actagatgtt |
| | ttggaggctg tgtgtttgcc |
| 1501 | tatgttggct gctataataa gcgtgcctac tgggttcctc |
| | gtgctagtgc tgatattggc |
| 1561 | tcaggccata ctggcattac tggtgacaat gtggagacct |
| | tgaatgagga tctccttgag |
| 1621 | atactgagtc gtgaacgtgt taacattaac attgttggcg |
| | attttcattt gaatgaagag |
| 1681 | gttgccatca ttttggcatc tttctctgct tctacaagtg |
| | cctttattga cactataaag |
| 1741 | agtcttgatt acaagtcttt caaaaccatt gttgagtcct |
| | gcggtaacta taaagttacc |
| 1801 | aagggaaagc ccgtaaaagg tgcttggaac attggacaac |
| | agagatcagt tttaacacca |
| 1861 | ctgtgtggtt ttccctcaca ggctgctggt gttatcagat |
| | caatttttgc gcgcacactt |
| 1921 | gatgcagcaa accactcaat tcctgatttg caaagagcag |
| | ctgtcaccat acttgatggt |
| 1981 | atttctgaac agtcattacg tcttgtcgac gccatggttt |
| | atacttcaga cctgctcacc |
| 2041 | aacagtgtca ttattatggc atatgtaact ggtggtcttg |
| | tacaacagac ttctcagtgg |
| 2101 | ttgtctaatc ttttgggcac tactgttgaa aaactcaggc |
| | ctatctttga atggattgag |
| 2161 | gcgaaactta gtgcaggagt tgaatttctc aaggatgctt |
| | gggagattct caaatttctc |
| 2221 | attacaggtg tttttgacat cgtcaagggt caaatacagg |
| | ttgcttcaga taacatcaag |
| 2281 | gattgtgtaa aatgcttcat tgatgttgtt aacaaggcac |
| | tcgaaatgtg cattgatcaa |
| 2341 | gtcactatcg ctggcgcaaa gttgcgatca ctcaacttag |
| | gtgaagtctt catcgctcaa |
| 2401 | agcaagggac tttaccgtca gtgtatacgt ggcaaggagc |
| | agctgcaact actcatgcct |
| 2461 | cttaaggcac caaaagaagt aacctttctt gaaggtgatt |
| | cacatgacac agtacttacc |
| 2521 | tctgaggagg ttgttctcaa gaacggtgaa ctcgaagcac |
| | tcgagacgcc cgttgatagc |
| 2581 | ttcacaaatg gagctatcgt tggcacacca gtctgtgtaa |
| | atggcctcat gctcttagag |
| 2641 | attaaggaca aagaacaata ctgcgcattg tctcctggtt |
| | tactggctac aaacaatgtc |
| 2701 | tttcgcttaa aagggggtgc accaattaaa ggtgtaacct |
| | ttggagaaga tactgtttgg |
| 2761 | gaagttcaag gttacaagaa tgtgagaatc acatttgagc |
| | ttgatgaacg tgttgacaaa |
| 2821 | gtgcttaatg aaaagtgctc tgtctacact gttgaatccg |
| | gtaccgaagt tactgagttt |
| 2881 | gcatgtgttg tagcagaggc tgttgtgaag actttacaac |
| | cagtttctga tctccttacc |
| 2941 | aacatgggta ttgatcttga tgagtggagt gtagctacat |
| | tctacttatt tgatgatgct |
| 3001 | ggtgaagaaa acttttcatc acgtatgtat tgttcctttt |
| | accctccaga tgaggaagaa |
| 3061 | gaggacgatg cagagtgtga ggaagaagaa attgatgaaa |
| | cctgtgaaca tgagtacggt |
| 3121 | acagaggatg attatcaagg tctccctctg gaatttggtg |
| | cctcagctga aacagttcga |
| 3181 | gttgaggaag aagaagagga agactggctg gatgatacta |
| | ctgagcaatc agagattgag |
| 3241 | ccagaaccag aacctacacc tgaagaacca gttaatcagt |
| | ttactggtta tttaaaactt |
| 3301 | actgacaatg ttgccattaa atgtgttgac atcgttaagg |
| | aggcacaaag tgctaatcct |
| 3361 | atggtgattg taaatgctgc taacatacac ctgaaacatg |
| | gtggtggtgt agcaggtgca |
| 3421 | ctcaacaagg caaccaatgg tgccatgcaa aaggagagtg |
| | atgattacat taagctaaat |
| 3481 | ggccctctta cagtaggagg gtcttgtttg ctttctggac |
| | ataatcttgc taagaagtgt |
| 3541 | ctgcatgttg ttggacctaa cctaaatgca ggtgaggaca |
| | tccagcttct taaggcagca |
| 3601 | tatgaaaatt tcaattcaca ggacatctta cttgcaccat |
| | tgttgtcagc aggcatattt |
| 3661 | ggtgctaaac cacttcagtc tttacaagtg tgcgtgcaga |
| | cggttcgtac acaggtttat |
| 3721 | attgcagtca atgacaaagc tctttatgag caggttgtca |
| | tggattatct tgataacctg |
| 3781 | aagcctagag tggaagcacc taaacaagag gagccaccaa |
| | acacagaaga ttccaaaact |
| 3841 | gaggagaaat ctgtcgtaca gaagcctgtc gatgtgaagc |
| | caaaaattaa ggcctgcatt |
| 3901 | gatgaggtta ccacaacact ggaagaaact aagtttctta |
| | ccaataagtt actcttgttt |
| 3961 | gctgatatca atggtaagct ttaccatgat tctcagaaca |
| | tgcttagagg tgaagatatg |
| 4021 | tctttccttg agaaggatgc accttacatg gtaggtgatg |
| | ttatcactag tggtgatatc |
| 4081 | acttgtgttg taataccctc caaaaaggct ggtggcacta |
| | ctgagatgct ctcaagagct |
| 4141 | ttgaagaaag tgccagttga tgagtatata accacgtacc |
| | ctggacaagg atgtgctggt |
| 4201 | tatacacttg aggaagctaa gactgctctt aagaaatgca |
| | aatctgcatt ttatgtacta |
| 4261 | ccttcagaag cacctaatgc taaggaagag attctaggaa |
| | ctgtatcctg gaatttgaga |
| 4321 | gaaatgcttg ctcatgctga agagacaaga aaattaatgc |
| | ctatatgcat ggatgttaga |
| 4381 | gccataatgg caaccatcca acgtaagtat aaaggaatta |
| | aaattcaaga gggcatcgtt |
| 4441 | gactatggtg tccgattctt cttttatact agtaaagagc |
| | ctgtagcttc tattattacg |
| 4501 | aagctgaact ctctaaatga gccgcttgtc acaatgccaa |
| | ttggttatgt gacacatggt |
| 4561 | tttaatcttg aagaggctgc gcgctgtatg cgttctctta |
| | aagctcctgc cgtagtgtca |
| 4621 | gtatcatcac cagatgctgt tactacatat aatggatacc |
| | tcacttcgtc atcaaagaca |
| 4681 | tctgaggagc actttgtaga aacagtttct ttggctggct |
| | cttacagaga ttggtcctat |
| 4741 | tcaggacagc gtacagagtt aggtgttgaa tttcttaagc |
| | gtggtgacaa aattgtgtac |
| 4801 | cacactctgg agagccccgt cgagtttcat cttgacggtg |
| | aggttctttc acttgacaaa |
| 4861 | ctaaagagtc tcttatccct gcgggaggtt aagactataa |
| | aagtgttcac aactgtggac |
| 4921 | aacactaatc tccacacaca gcttgtggat atgtctatga |
| | catatggaca gcagtttggt |
| 4981 | ccaacatact tggatggtgc tgatgttaca aaaattaaac |
| | ctcatgtaaa tcatgagggt |
| 5041 | aagactttct ttgtactacc tagtgatgac acactacgta |
| | gtgaagcttt cgagtactac |
| 5101 | catactcttg atgagagttt tcttggtagg tacatgtctg |
| | ctttaaacca cacaaagaaa |
| 5161 | tggaaatttc ctcaagttgg tggtttaact tcaattaaat |
| | gggctgataa caattgttat |
| 5221 | ttgtctagtg ttttattagc acttcaacag cttgaagtca |
| | aattcaatgc accagcactt |
| 5281 | caagaggctt attatagagc ccgtgctggt gatgctgcta |
| | acttttgtgc actcatactc |
| 5341 | gcttacagta ataaaactgt tggcgagctt ggtgatgtca |
| | gagaaactat gacccatctt |
| 5401 | ctacagcatg ctaatttgga atctgcaaag cgagttctta |
| | atgtggtgtg taaacattgt |
| 5461 | ggtcagaaaa ctactacctt aacgggtgta gaagctgtga |
| | tgtatatggg tactctatct |
| 5521 | tatgataatc ttaagacagg tgtttccatt ccatgtgtgt |
| | gtggtcgtga tgctacacaa |
| 5581 | tatctagtac aacaagagtc ttcttttgtt atgatgtctg |
| | caccacctgc tgagtataaa |
| 5641 | ttacagcaag gtacattctt atgtgcgaat gagtacactg |
| | gtaactatca gtgtggtcat |
| 5701 | tacactcata taactgctaa ggagaccctc tatcgtattg |
| | acggagctca ccttacaaag |
| 5761 | atgtcagagt acaaaggacc agtgactgat gttttctaca |
| | aggaaacatc ttacactaca |
| 5821 | accatcaagc ctgtgtcgta taaactcgat ggagttactt |
| | acacagagat tgaaccaaaa |
| 5881 | ttggatgggt attataaaaa ggataatgct tactatacag |
| | agcagcctat agaccttgta |
| 5941 | ccaactcaac cattaccaaa tgcgagtttt gataatttca |
| | aactcacatg ttctaacaca |
| 6001 | aaatttgctg atgatttaaa tcaaatgaca ggcttcacaa |
| | agccagcttc acgagagcta |
| 6061 | tctgtcacat tcttcccaga cttgaatggc gatgtagtgg |
| | ctattgacta tagacactat |
| 6121 | tcagcgagtt tcaagaaagg tgctaaatta ctgcataagc |
| | caattgtttg gcacattaac |
| 6181 | caggctacaa ccaagacaac gttcaaacca aacacttggt |
| | gtttacgttg tctttggagt |
| 6241 | acaaagccag tagatacttc aaattcattt gaagttctgg |
| | cagtagaaga cacacaagga |
| 6301 | atggacaatc ttgcttgtga aagtcaacaa cccacctctg |
| | aagaagtagt ggaaaatcct |
| 6361 | accatacaga aggaagtcat agagtgtgac gtgaaaacta |
| | ccgaagttgt aggcaatgtc |
| 6421 | atacttaaac catcagatga aggtgttaaa gtaacacaag |
| | agttaggtca tgaggatctt |
| 6481 | atggctgctt atgtggaaaa cacaagcatt accattaaga |
| | aacctaatga gctttcacta |
| 6541 | gccttaggtt taaaaacaat tgccactcat ggtattgctg |
| | caattaatag tgttccttgg |
| 6601 | agtaaaattt tggcttatgt caaaccattc ttaggacaag |
| | cagcaattac aacatcaaat |
| 6661 | tgcgctaaga gattagcaca acgtgtgttt aacaattata |
| | tgccttatgt gtttacatta |
| 6721 | ttgttccaat tgtgtacttt tactaaaagt accaattcta |
| | gaattagagc ttcactacct |
| 6781 | acaactattg ctaaaaatag tgttaagagt gttgctaaat |
| | tatgtttgga tgccggcatt |
| 6841 | aattatgtga agtcacccaa attttctaaa ttgttcacaa |
| | tcgctatgtg gctattgttg |
| 6901 | ttaagtattt gcttaggttc tctaatctgt gtaactgctg |
| | cttttggtgt actcttatct |
| 6961 | aattttggtg ctccttctta ttgtaatggc gttagagaat |
| | tgtatcttaa ttcgtctaac |
| 7021 | gttactacta tggatttctg tgaaggttct tttccttgca |
| | gcatttgttt aagtggatta |
| 7081 | gactcccttg attcttatcc agctcttgaa accattcagg |
| | tgacgatttc atcgtacaag |
| 7141 | ctagacttga caattttagg tctggccgct gagtgggttt |
| | tggcatatat gttgttcaca |
| 7201 | aaattctttt atttattagg tctttcagct ataatgcagg |
| | tgttctttgg ctattttgct |
| 7261 | agtcatttca tcagcaattc ttggctcatg tggtttatca |
| | ttagtattgt acaaatggca |
| 7321 | cccgtttctg caatggttag gatgtacatc ttctttgctt |
| | ctttctacta catatggaag |
| 7381 | agctatgttc atatcatgga tggttgcacc tcttcgactt |
| | gcatgatgtg ctataagcgc |
| 7441 | aatcgtgcca cacgcgttga gtgtacaact attgttaatg |
| | gcatgaagag atctttctat |
| 7501 | gtctatgcaa atggaggccg tggcttctgc aagactcaca |
| | attggaattg tctcaattgt |
| 7561 | gacacatttt gcactggtag tacattcatt agtgatgaag |
| | ttgctcgtga tttgtcactc |
| 7621 | cagtttaaaa gaccaatcaa ccctactgac cagtcatcgt |
| | atattgttga tagtgttgct |
| 7681 | gtgaaaaatg gcgcgcttca cctctacttt gacaaggctg |
| | gtcaaaagac ctatgagaga |
| 7741 | catccgctct cccattttgt caatttagac aatttgagag |
| | ctaacaacac taaaggttca |
| 7801 | ctgcctatta atgtcatagt ttttgatggc aagtccaaat |
| | gcgacgagtc tgcttctaag |
| 7861 | tctgcttctg tgtactacag tcagctgatg tgccaaccta |
| | ttctgttgct tgaccaagct |
| 7921 | cttgtatcag acgttggaga tagtactgaa gtttccgtta |
| | agatgtttga tgcttatgtc |
| 7981 | gacacctttt cagcaacttt tagtgttcct atggaaaaac |
| | ttaaggcact tgttgctaca |
| 8041 | gctcacagcg agttagcaaa gggtgtagct ttagatggtg |
| | tcctttctac attcgtgtca |
| 8101 | gctgcccgac aaggtgttgt tgataccgat gttgacacaa |
| | aggatgttat tgaatgtctc |
| 8161 | aaactttcac atcactctga cttagaagtg acaggtgaca |
| | gttgtaacaa tttcatgctc |
| 8221 | acctataata aggttgaaaa catgacgccc agagatcttg |
| | gcgcatgtat tgactgtaat |
| 8281 | gcaaggcata tcaatgccca agtagcaaaa agtcacaatg |
| | tttcactcat ctggaatgta |
| 8341 | aaagactaca tgtctttatc tgaacagctg cgtaaacaaa |
| | ttcgtagtgc tgccaagaag |
| 8401 | aacaacatac cttttagact aacttgtgct acaactagac |
| | aggttgtcaa tgtcataact |
| 8461 | actaaaatct cactcaaggg tggtaagatt gttagtactt |
| | gttttaaact tatgcttaag |
| 8521 | gccacattat tgtgcgttct tgctgcattg gtttgttata |
| | tcgttatgcc agtacataca |
| 8581 | ttgtcaatcc atgatggtta cacaaatgaa atcattggtt |
| | acaaagccat tcaggatggt |
| 8641 | gtcactcgtg acatcatttc tactgatgat tgttttgcaa |
| | ataaacatgc tggttttgac |
| 8701 | gcatggttta gccagcgtgg tggttcatac aaaaatgaca |
| | aaagctgccc tgtagtagct |
| 8761 | gctatcatta caagagagat tggtttcata gtgcctggct |
| | taccgggtac tgtgctgaga |
| 8821 | gcaatcaatg gtgacttctt gcattttcta cctcgtgttt |
| | ttagtgctgt tggcaacatt |
| 8881 | tgctacacac cttccaaact cattgagtat agtgattttg |
| | ctacctctgc ttgcgttctt |
| 8941 | gctgctgagt gtacaatttt taaggatgct atgggcaaac |
| | ctgtgccata ttgttatgac |
| 9001 | actaatttgc tagagggttc tatttcttat agtgagcttc |
| | gtccagacac tcgttatgtg |
| 9061 | cttatggatg gttccatcat acagtttcct aacacttacc |
| | tggagggttc tgttagagta |
| 9121 | gtaacaactt ttgatgctga gtactgtaga catggtacat |
| | gcgaaaggtc agaagtaggt |
| 9181 | atttgcctat ctaccagtgg tagatgggtt cttaataatg |
| | agcattacag agctctatca |
| 9241 | ggagttttct gtggtgttga tgcgatgaat ctcatagcta |
| | acatctttac tcctcttgtg |
| 9301 | caacctgtgg gtgctttaga tgtgtctgct tcagtagtgg |
| | ctggtggtat tattgccata |
| 9361 | ttggtgactt gtgctgccta ctactttatg aaattcagac |
| | gtgtttttgg tgagtacaac |
| 9421 | catgttgttg ctgctaatgc acttttgttt ttgatgtctt |
| | tcactatact ctgtctggta |
| 9481 | ccagcttaca gctttctgcc gggagtctac tcagtctttt |
| | acttgtactt gacattctat |
| 9541 | ttcaccaatg atgtttcatt cttggctcac cttcaatggt |
| | ttgccatgtt ttctcctatt |
| 9601 | gtgccttttt ggataacagc aatctatgta ttctgtattt |
| | ctctgaagca ctgccattgg |
| 9661 | ttctttaaca actatcttag gaaaagagtc atgtttaatg |
| | gagttacatt tagtaccttc |
| 9721 | gaggaggctg ctttgtgtac ctttttgctc aacaaggaaa |
| | tgtacctaaa attgcgtagc |
| 9781 | gagacactgt tgccacttac acagtataac aggtatcttg |
| | ctctatataa caagtacaag |
| 9841 | tatttcagtg gagccttaga tactaccagc tatcgtgaag |
| | cagcttgctg ccacttagca |
| 9901 | aaggctctaa atgactttag caactcaggt gctgatgttc |
| | tctaccaacc accacagaca |
| 9961 | tcaatcactt ctgctgttct gcagagtggt tttaggaaaa |
| | tggcattccc gtcaggcaaa |
| 10021 | gttgaagggt gcatggtaca agtaacctgt ggaactacaa |
| | ctcttaatgg attgtggttg |
| 10081 | gatgacacag tatactgtcc aagacatgtc atttgcacag |
| | cagaagacat gcttaatcct |
| 10141 | aactatgaag atctgctcat tcgcaaatcc aaccatagct |
| | ttcttgttca ggctggcaat |
| 10201 | gttcaacttc gtgttattgg ccattctatg caaaattgtc |
| | tgcttaggct taaagttgat |
| 10261 | acttctaacc ctaagacacc caagtataaa tttgtccgta |
| | tccaacctgg tcaaacattt |
| 10321 | tcagttctag catgctacaa tggttcacca tctggtgttt |
| | atcagtgtgc catgagacct |
| 10381 | aatcatacca ttaaaggttc tttccttaat ggatcatgtg |
| | gtagtgttgg ttttaacatt |
| 10441 | gattatgatt gcgtgtcttt ctgctatatg catcatatgg |
| | agcttccaac aggagtacac |
| 10501 | gctggtactg acttagaagg taaattctat ggtccatttg |
| | ttgacagaca aactgcacag |
| 10561 | gctgcaggta cagacacaac cataacatta aatgttttgg |
| | catggctgta tgctgctgtt |
| 10621 | atcaatggtg ataggtggtt tcttaataga ttcaccacta |
| | ctttgaatga ctttaacctt |
| 10681 | gtggcaatga agtacaacta tgaacctttg acacaagatc |
| | atgttgacat attgggacct |
| 10741 | ctttctgctc aaacaggaat tgccgtctta gatatgtgtg |
| | ctgctttgaa agagctgctg |
| 10801 | cagaatggta tgaatggtcg tactatcctt ggtagcacta |
| | ttttagaaga tgagtttaca |
| 10861 | ccatttgatg ttgttagaca atgctctggt gttaccttcc |
| | aaggtaagtt caagaaaatt |
| 10921 | gttaagggca ctcatcattg gatgctttta actttcttga |
| | catcactatt gattcttgtt |
| 10981 | caaagtacac agtggtcact gtttttcttt gtttacgaga |
| | atgctttctt gccatttact |
| 11041 | cttggtatta tggcaattgc tgcatgtgct atgctgcttg |
| | ttaagcataa gcacgcattc |
| 11101 | ttgtgcttgt ttctgttacc ttctcttgca acagttgctt |
| | actttaatat ggtctacatg |
| 11161 | cctgctagct gggtgatgcg tatcatgaca tggcttgaat |
| | tggctgacac tagcttgtct |
| 11221 | ggttataggc ttaaggattg tgttatgtat gcttcagctt |
| | tagttttgct tattctcatg |
| 11281 | acagctcgca ctgtttatga tgatgctgct agacgtgttt |
| | ggacactgat gaatgtcatt |
| 11341 | acacttgttt acaaagtcta ctatggtaat gctttagatc |
| | aagctatttc catgtgggcc |
| 11401 | ttagttattt ctgtaacctc taactattct ggtgtcgtta |
| | cgactatcat gtttttagct |
| 11461 | agagctatag tgtttgtgtg tgttgagtat tacccattgt |
| | tatttattac tggcaacacc |
| 11521 | ttacagtgta tcatgcttgt ttattgtttc ttaggctatt |
| | gttgctgctg ctactttggc |
| 11581 | cttttctgtt tactcaaccg ttacttcagg cttactcttg |
| | gtgtttatga ctacttggtc |
| 11641 | tctacacaag aatttaggta tatgaactcc caggggcttt |
| | tgcctcctaa gagtagtatt |
| 11701 | gatgctttca agcttaacat taagttgttg ggtattggag |
| | gtaaaccatg tatcaaggtt |
| 11761 | gctactgtac agtctaaaat gtctgacgta aagtgcacat |
| | ctgtggtact gctctcggtt |
| 11821 | cttcaacaac ttagagtaga gtcatcttct aaattgtggg |
| | cacaatgtgt acaactccac |
| 11881 | aatgatattc ttcttgcaaa agacacaact gaagctttcg |
| | agaagatggt ttctcttttg |
| 11941 | tctgttttgc tatccatgca gggtgctgta gacattaata |
| | ggttgtgcga ggaaatgctc |
| 12001 | gataaccgtg ctactcttca ggctattgct tcagaattta |
| | gttctttacc atcatatgcc |
| 12061 | gcttatgcca ctgcccagga ggcctatgag caggctgtag |
| | ctaatggtga ttctgaagtc |
| 12121 | gttctcaaaa agttaaagaa atctttgaat gtggctaaat |
| | ctgagtttga ccgtgatgct |
| 12181 | gccatgcaac gcaagttgga aaagatggca gatcaggcta |
| | tgacccaaat gtacaaacag |
| 12241 | gcaagatctg aggacaagag ggcaaaagta actagtgcta |
| | tgcaaacaat gctcttcact |
| 12301 | atgcttagga agcttgataa tgatgcactt aacaacatta |
| | tcaacaatgc gcgtgatggt |
| 12361 | tgtgttccac tcaacatcat accattgact acagcagcca |
| | aactcatggt tgttgtccct |
| 12421 | gattatggta cctacaagaa cacttgtgat ggtaacacct |
| | ttacatatgc atctgcactc |
| 12481 | tgggaaatcc agcaagttgt tgatgcggat agcaagattg |
| | ttcaacttag tgaaattaac |
| 12541 | atggacaatt caccaaattt ggcttggcct cttattgtta |
| | cagctctaag agccaactca |
| 12601 | gctgttaaac tacagaataa tgaactgagt ccagtagcac |
| | tacgacagat gtcctgtgcg |
| 12661 | gctggtacca cacaaacagc ttgtactgat gacaatgcac |
| | ttgcctacta taacaattcg |
| 12721 | aagggaggta ggtttgtgct ggcattacta tcagaccacc |
| | aagatctcaa atgggctaga |
| 12781 | ttccctaaga gtgatggtac aggtacaatt tacacagaac |
| | tggaaccacc ttgtaggttt |
| 12841 | gttacagaca caccaaaagg gcctaaagtg aaatacttgt |
| | acttcatcaa aggcttaaac |
| 12901 | aacctaaata gaggtatggt gctgggcagt ttagctgcta |
| | cagtacgtct tcaggctgga |
| 12961 | aatgctacag aagtacctgc caattcaact gtgctttcct |
| | tctgtgcttt tgcagtagac |
| 13021 | cctgctaaag catataagga ttacctagca agtggaggac |
| | aaccaatcac caactgtgtg |
| 13081 | aagatgttgt gtacacacac tggtacagga caggcaatta |
| | ctgtaacacc agaagctaac |
| 13141 | atggaccaag agtcctttgg tggtgcttca tgttgtctgt |
| | attgtagatg ccacattgac |
| 13201 | catccaaatc ctaaaggatt ctgtgacttg aaaggtaagt |
| | acgtccaaat acctaccact |
| 13261 | tgtgctaatg acccagtggg ttttacactt agaaacacag |
| | tctgtaccgt ctgcggaatg |
| 13321 | tggaaaggtt atggctgtag ttgtgaccaa ctccgcgaac |
| | ccttgatgca gtctgcggat |
| 13381 | gcatcaacgt ttttaaacgg gtttgcggtg taagtgcagc |
| | ccgtcttaca ccgtgcggca |
| 13441 | caggcactag tactgatgtc gtctacaggg cttttgatat |
| | ttacaacgaa aaagttgctg |
| 13501 | gttttgcaaa gttcctaaaa actaattgct gtcgcttcca |
| | ggagaaggat gaggaaggca |
| 13561 | atttattaga ctcttacttt gtagttaaga ggcatactat |
| | gtctaactac caacatgaag |
| 13621 | agactattta taacttggtt aaagattgtc cagcggttgc |
| | tgtccatgac tttttcaagt |
| 13681 | ttagagtaga tggtgacatg gtaccacata tatcacgtca |
| | gcgtctaact aaatacacaa |
| 13741 | tggctgattt agtctatgct ctacgtcatt ttgatgaggg |
| | taattgtgat acattaaaag |
| 13801 | aaatactcgt cacatacaat tgctgtgatg atgattattt |
| | caataagaag gattggtatg |
| 13861 | acttcgtaga gaatcctgac atcttacgcg tatatgctaa |
| | cttaggtgag cgtgtacgcc |
| 13921 | aatcattatt aaagactgta caattctgcg atgctatgcg |
| | tgatgcaggc attgtaggcg |
| 13981 | tactgacatt agataatcag gatcttaatg ggaactggta |
| | cgatttcggt gatttcgtac |
| 14041 | aagtagcacc aggctgcgga gttcctattg tggattcata |
| | ttactcattg ctgatgccca |
| 14101 | tcctcacttt gactagggca ttggctgctg agtcccatat |
| | ggatgctgat ctcgcaaaac |
| 14161 | cacttattaa gtgggatttg ctgaaatatg attttacgga |
| | agagagactt tgtctcttcg |
| 14221 | accgttattt taaatattgg gaccagacat accatcccaa |
| | ttgtattaac tgtttggatg |
| 14281 | ataggtgtat ccttcattgt gcaaacttta atgtgttatt |
| | ttctactgtg tttccaccta |
| 14341 | caagttttgg accactagta agaaaaatat ttgtagatgg |
| | tgttcctttt gttgtttcaa |
| 14401 | ctggatacca ttttcgtgag ttaggagtcg tacataatca |
| | ggatgtaaac ttacatagct |
| 14461 | cgcgtctcag tttcaaggaa cttttagtgt atgctgctga |
| | tccagctatg catgcagctt |
| 14521 | ctggcaattt attgctagat aaacgcacta catgcttttc |
| | agtagctgca ctaacaaaca |
| 14581 | atgttgcttt tcaaactgtc aaacccggta attttaataa |
| | agacttttat gactttgctg |
| 14641 | tgtctaaagg tttctttaag gaaggaagtt ctgttgaact |
| | aaaacacttc ttctttgctc |
| 14701 | aggatggcaa cgctgctatc agtgattatg actattatcg |
| | ttataatctg ccaacaatgt |
| 14761 | gtgatatcag acaactccta ttcgtagttg aagttgttga |
| | taaatacttt gattgttacg |
| 14821 | atggtggctg tattaatgcc aaccaagtaa tcgttaacaa |
| | tctggataaa tcagctggtt |
| 14881 | tcccatttaa taaatggggt aaggctagac tttattatga |
| | ctcaatgagt tatgaggatc |
| 14941 | aagatgcact tttcgcgtat actaagcgta atgtcatccc |
| | tactataact caaatgaatc |
| 15001 | ttaagtatgc cattagtgca aagaatagag ctcgcaccgt |
| | agctggtgtc tctatctgta |
| 15061 | gtactatgac aaatagacag tttcatcaga aattattgaa |
| | gtcaatagcc gccactagag |
| 15121 | gagctactgt ggtaattgga acaagcaagt tttacggtgg |
| | ctggcataat atgttaaaaa |
| 15181 | ctgtttacag tgatgtagaa actccacacc ttatgggttg |
| | ggattatcca aaatgtgaca |
| 15241 | gagccatgcc taacatgctt aggataatgg cctctcttgt |
| | tcttgctcgc aaacataaca |
| 15301 | cttgctgtaa cttatcacac cgtttctaca ggttagctaa |
| | cgagtgtgcg caagtattaa |
| 15361 | gtgagatggt catgtgtggc ggctcactat atgttaaacc |
| | aggtggaaca tcatccggtg |
| 15421 | atgctacaac tgcttatgct aatagtgtct ttaacatttg |
| | tcaagctgtt acagccaatg |
| 15481 | taaatgcact tctttcaact gatggtaata agatagctga |
| | caagtatgtc cgcaatctac |
| 15541 | aacacaggct ctatgagtgt ctctatagaa atagggatgt |
| | tgatcatgaa ttcgtggatg |
| 15601 | agttttacgc ttacctgcgt aaacatttct ccatgatgat |
| | tctttctgat gatgccgttg |
| 15661 | tgtgctataa cagtaactat gcggctcaag gtttagtagc |
| | tagcattaag aactttaagg |
| 15721 | cagttcttta ttatcaaaat aatgtgttca tgtctgaggc |
| | aaaatgttgg actgagactg |
| 15781 | accttactaa aggacctcac gaattttgct cacagcatac |
| | aatgctagtt aaacaaggag |
| 15841 | atgattacgt gtacctgcct tacccagatc catcaagaat |
| | attaggcgca ggctgttttg |
| 15901 | tcgatgatat tgtcaaaaca gatggtacac ttatgattga |
| | aaggttcgtg tcactggcta |
| 15961 | ttgatgctta cccacttaca aaacatccta atcaggagta |
| | tgctgatgtc tttcacttgt |
| 16021 | atttacaata cattagaaag ttacatgatg agcttactgg |
| | ccacatgttg gacatgtatt |
| 16081 | ccgtaatgct aactaatgat aacacctcac ggtactggga |
| | acctgagttt tatgaggcta |
| 16141 | tgtacacacc acatacagtc ttgcaggctg taggtgcttg |
| | tgtattgtgc aattcacaga |
| 16201 | cttcacttcg ttgcggtgcc tgtattagga gaccattcct |
| | atgttgcaag tgctgctatg |
| 16261 | accatgtcat ttcaacatca cacaaattag tgttgtctgt |
| | taatccctat gtttgcaatg |
| 16321 | ccccaggttg tgatgtcact gatgtgacac aactgtatct |
| | aggaggtatg agctattatt |
| 16381 | gcaagtcaca taagcctccc attagttttc cattatgtgc |
| | taatggtcag gtttttggtt |
| 16441 | tatacaaaaa cacatgtgta ggcagtgaca atgtcactga |
| | cttcaatgcg atagcaacat |
| 16501 | gtgattggac taatgctggc gattacatac ttgccaacac |
| | ttgtactgag agactcaagc |
| 16561 | ttttcgcagc agaaacgctc aaagccactg aggaaacatt |
| | taagctgtca tatggtattg |
| 16621 | ccactgtacg cgaagtactc tctgacagag aattgcatct |
| | ttcatgggag gttggaaaac |
| 16681 | ctagaccacc attgaacaga aactatgtct ttactggtta |
| | ccgtgtaact aaaaatagta |
| 16741 | aagtacagat tggagagtac acctttgaaa aaggtgacta |
| | tggtgatgct gttgtgtaca |
| 16801 | gaggtactac gacatacaag ttgaatgttg gtgattactt |
| | tgtgttgaca tctcacactg |
| 16861 | taatgccact tagtgcacct actctagtgc cacaagagca |
| | ctatgtgaga attactggct |
| 16921 | tgtacccaac actcaacatc tcagatgagt tttctagcaa |
| | tgttgcaaat tatcaaaagg |
| 16981 | tcggcatgca aaagtactct acactccaag gaccacctgg |
| | tactggtaag agtcattttg |
| 17041 | ccatcggact tgctctctat tacccatctg ctcgcatagt |
| | gtatacggca tgctctcatg |
| 17101 | cagctgttga tgccctatgt gaaaaggcat taaaatattt |
| | gcccatagat aaatgtagta |
| 17161 | gaatcatacc tgcgcgtgcg cgcgtagagt gttttgataa |
| | attcaaagtg aattcaacac |
| 17221 | tagaacagta tgttttctgc actgtaaatg cattgccaga |
| | aacaactgct gacattgtag |
| 17281 | tctttgatga aatctctatg gctactaatt atgacttgag |
| | tgttgtcaat gctagacttc |
| 17341 | gtgcaaaaca ctacgtctat attggcgatc ctgctcaatt |
| | accagccccc cgcacattgc |
| 17401 | tgactaaagg cacactagaa ccagaatatt ttaattcagt |
| | gtgcagactt atgaaaacaa |
| 17461 | taggtccaga catgttcctt ggaacttgtc gccgttgtcc |
| | tgctgaaatt gttgacactg |
| 17521 | tgagtgcttt agtttatgac aataagctaa aagcacacaa |
| | ggataagtca gctcaatgct |
| 17581 | tcaaaatgtt ctacaaaggt gttattacac atgatgtttc |
| | atctgcaatc aacagacctc |
| 17641 | aaataggcgt tgtaagagaa tttcttacac gcaatcctgc |
| | ttggagaaaa gctgttttta |
| 17701 | tctcacctta taattcacag aacgctgtag cttcaaaaat |
| | cttaggattg cctacgcaga |
| 17761 | ctgttgattc atcacagggt tctgaatatg actatgtcat |
| | attcacacaa actactgaaa |
| 17821 | cagcacactc ttgtaatgtc aaccgcttca atgtggctat |
| | cacaagggca aaaattggca |
| 17881 | ttttgtgcat aatgtctgat agagatcttt atgacaaact |
| | gcaatttaca agtctagaaa |
| 17941 | taccacgtcg caatgtggct acattacaag cagaaaatgt |
| | aactggactt tttaaggact |
| 18001 | gtagtaagat cattactggt cttcatccta cacaggcacc |
| | tacacacctc agcgttgata |
| 18061 | taaagttcaa gactgaagga ttatgtgttg acataccagg |
| | cataccaaag gacatgacct |
| 18121 | accgtagact catctctatg atgggtttca aaatgaatta |
| | ccaagtcaat ggttacccta |
| 18181 | atatgtttat cacccgcgaa gaagctattc gtcacgttcg |
| | tgcgtggatt ggctttgatg |
| 18241 | tagagggctg tcatgcaact agagatgctg tgggtactaa |
| | cctacctctc cagctaggat |
| 18301 | tttctacagg tgttaactta gtagctgtac cgactggtta |
| | tgttgacact gaaaataaca |
| 18361 | cagaattcac cagagttaat gcaaaacctc caccaggtga |
| | ccagtttaaa catcttatac |
| 18421 | cactcatgta taaaggcttg ccctggaatg tagtgcgtat |
| | taagatagta caaatgctca |
| 18481 | gtgatacact gaaaggattg tcagacagag tcgtgttcgt |
| | cctttgggcg catggctttg |
| 18541 | agcttacatc aatgaagtac tttgtcaaga ttggacctga |
| | aagaacgtgt tgtctgtgtg |
| 18601 | acaaacgtgc aacttgcttt tctacttcat cagatactta |
| | tgcctgctgg aatcattctg |
| 18661 | tgggttttga ctatgtctat aacccattta tgattgatgt |
| | tcagcagtgg ggctttacgg |
| 18721 | gtaaccttca gagtaaccat gaccaacatt gccaggtaca |
| | tggaaatgca catgtggcta |
| 18781 | gttgtgatgc tatcatgact agatgtttag cagtccatga |
| | gtgctttgtt aagcgcgttg |
| 18841 | attggtctgt tgaataccct attataggag atgaactgag |
| | ggttaattct gcttgcagaa |
| 18901 | aagtacaaca catggttgtg aagtctgcat tgcttgctga |
| | taagtttcca gttcttcatg |
| 18961 | acattggaaa tccaaaggct atcaagtgtg tgcctcaggc |
| | tgaagtagaa tggaagttct |
| 19021 | acgatgctca gccatgtagt gacaaagctt acaaaataga |
| | ggaactcttc tattcttatg |
| 19081 | ctacacatca cgataaattc actgatggtg tttgtttgtt |
| | ttggaattgt aacgttgatc |
| 19141 | gttacccagc caatgcaatt gtgtgtaggt ttgacacaag |
| | agtcttgtca aacttgaact |
| 19201 | taccaggctg tgatggtggt agtttgtatg tgaataagca |
| | tgcattccac actccagctt |
| 19261 | tcgataaaag tgcatttact aatttaaagc aattgccttt |
| | cttttactat tctgatagtc |
| 19321 | cttgtgagtc tcatggcaaa caagtagtgt cggatattga |
| | ttatgttcca ctcaaatctg |
| 19381 | ctacgtgtat tacacgatgc aatttaggtg gtgctgtttg |
| | cagacaccat gcaaatgagt |
| 19441 | accgacagta cttggatgca tataatatga tgatttctgc |
| | tggatttagc ctatggattt |
| 19501 | acaaacaatt tgatacttat aacctgtgga atacatttac |
| | caggttacag agtttagaaa |
| 19561 | atgtggctta taatgttgtt aataaaggac actttgatgg |
| | acacgccggc gaagcacctg |
| 19621 | tttccatcat taataatgct gtttacacaa aggtagatgg |
| | tattgatgtg gagatctttg |
| 19681 | aaaataagac aacacttcct gttaatgttg catttgagct |
| | ttgggctaag cgtaacatta |
| 19741 | aaccagtgcc agagattaag atactcaata atttgggtgt |
| | tgatatcgct gctaatactg |
| 19801 | taatctggga ctacaaaaga gaagccccag cacatgtatc |
| | tacaataggt gtctgcacaa |
| 19861 | tgactgacat tgccaagaaa cctactgaga gtgcttgttc |
| | ttcacttact gtcttgtttg |
| 19921 | atggtagagt ggaaggacag gtagaccttt ttagaaacgc |
| | ccgtaatggt gttttaataa |
| 19981 | cagaaggttc agtcaaaggt ctaacacctt caaagggacc |
| | agcacaagct agcgtcaatg |
| 20041 | gagtcacatt aattggagaa tcagtaaaaa cacagtttaa |
| | ctactttaag aaagtagacg |
| 20101 | gcattattca acagttgcct gaaacctact ttactcagag |
| | cagagactta gaggatttta |
| 20161 | agcccagatc acaaatggaa actgactttc tcgagctcgc |
| | tatggatgaa ttcatacagc |
| 20221 | gatataagct cgagggctat gccttcgaac acatcgttta |
| | tggagatttc agtcatggac |
| 20281 | aacttggcgg tcttcattta atgataggct tagccaagcg |
| | ctcacaagat tcaccactta |
| 20341 | aattagagga ttttatccct atggacagca cagtgaaaaa |
| | ttacttcata acagatgcgc |
| 20401 | aaacaggttc atcaaaatgt gtgtgttctg tgattgatct |
| | tttacttgat gactttgtcg |
| 20461 | agataataaa gtcacaagat ttgtcagtga tttcaaaagt |
| | ggtcaaggtt acaattgact |
| 20521 | atgctgaaat ttcattcatg ctttggtgta aggatggaca |
| | tgttgaaacc ttctacccaa |
| 20581 | aactacaagc aagtcaagcg tggcaaccag gtgttgcgat |
| | gcctaacttg tacaagatgc |
| 20641 | aaagaatgct tcttgaaaag tgtgaccttc agaattatgg |
| | tgaaaatgct gttataccaa |
| 20701 | aaggaataat gatgaatgtc gcaaagtata ctcaactgtg |
| | tcaatactta aatacactta |
| 20761 | ctttagctgt accctacaac atgagagtta ttcactttgg |
| | tgctggctct gataaaggag |
| 20821 | ttgcaccagg tacagctgtg ctcagacaat ggttgccaac |
| | tggcacacta cttgtcgatt |
| 20881 | cagatcttaa tgacttcgtc tccgacgcag attctacttt |
| | aattggagac tgtgcaacag |
| 20941 | tacatacggc taataaatgg gaccttatta ttagcgatat |
| | gtatgaccct aggaccaaac |
| 21001 | atgtgacaaa agagaatgac tctaaagaag ggtttttcac |
| | ttatctgtgt ggatttataa |
| 21061 | agcaaaaact agccctgggt ggttctatag ctgtaaagat |
| | aacagagcat tcttggaatg |
| 21121 | ctgaccttta caagcttatg ggccatttct catggtggac |
| | agcttttgtt acaaatgtaa |
| 21181 | atgcatcatc atcggaagca tttttaattg gggctaacta |
| | tcttggcaag ccgaaggaac |
| 21241 | aaattgatgg ctataccatg catgctaact acattttctg |
| | gaggaacaca aatcctatcc |
| 21301 | agttgtcttc ctattcactc tttgacatga gcaaatttcc |
| | tcttaaatta agaggaactg |
| 21361 | ctgtaatgtc tcttaaggag aatcaaatca atgatatgat |
| | ttattctctt ctggaaaaag |
| 21421 | gtaggcttat cattagagaa aacaacagag ttgtggtttc |
| | aagtgatatt cttgttaaca |
| 21481 | actaaacgaa catgtttatt ttcttattat ttcttactct |
| | cactagtggt agtgaccttg |
| 21541 | accggtgcac cacttttgat gatgttcaag ctcctaatta |
| | cactcaacat acttcatcta |
| 21601 | tgaggggggt ttactatcct gatgaaattt ttagatcaga |
| | cactctttat ttaactcagg |
| 21661 | atttatttct tccattttat tctaatgtta cagggtttca |
| | tactattaat catacgtttg |
| 21721 | gcaaccctgt catacctttt aaggatggta tttattttgc |
| | tgccacagag aaatcaaatg |
| 21781 | ttgtccgtgg ttgggttttt ggttctacca tgaacaacaa |
| | gtcacagtcg gtgattatta |
| 21841 | ttaacaattc tactaatgtt gttatacgag catgtaactt |
| | tgaattgtgt gacaaccctt |
| 21901 | tctttgctgt ttctaaaccc atgggtacac agacacatac |
| | tatgatattc gataatgcat |
| 21961 | ttaattgcac tttcgagtac atatctgatg ccttttcgct |
| | tgatgtttca gaaaagtcag |
| 22021 | gtaattttaa acacttacga gagtttgtgt ttaaaaataa |
| | agatgggttt ctctatgttt |
| 22081 | ataagggcta tcaacctata gatgtagttc gtgatctacc |
| | ttctggtttt aacactttga |
| 22141 | aacctatttt taagttgcct cttggtatta acattacaaa |
| | ttttagagcc attcttacag |
| 22201 | ccttttcacc tgctcaagac atttggggca cgtcagctgc |
| | agcctatttt gttggctatt |
| 22261 | taaagccaac tacatttatg ctcaagtatg atgaaaatgg |
| | tacaatcaca gatgctgttg |
| 22321 | attgttctca aaatccactt gctgaactca aatgctctgt |
| | taagagcttt gagattgaca |
| 22381 | aaggaattta ccagacctct aatttcaggg ttgttccctc |
| | aggagatgtt gtgagattcc |
| 22441 | ctaatattac aaacttgtgt ccttttggag aggtttttaa |
| | tgctactaaa ttcccttctg |
| 22501 | tctatgcatg ggagagaaaa aaaatttcta attgtgttgc |
| | tgattactct gtgctctaca |
| 22561 | actcaacatt tttttcaacc tttaagtgct atggcgtttc |
| | tgccactaag ttgaatgatc |
| 22621 | tttgcttctc caatgtctat gcagattctt ttgtagtcaa |
| | gggagatgat gtaagacaaa |
| 22681 | tagcgccagg acaaactggt gttattgctg attataatta |
| | taaattgcca gatgatttca |
| 22741 | tgggttgtgt ccttgcttgg aatactagga acattgatgc |
| | tacttcaact ggtaattata |
| 22801 | attataaata taggtatctt agacatggca agcttaggcc |
| | ctttgagaga gacatatcta |
| 22861 | atgtgccttt ctcccctgat ggcaaacctt gcaccccacc |
| | tgctcttaat tgttattggc |
| 22921 | cattaaatga ttatggtttt tacaccacta ctggcattgg |
| | ctaccaacct tacagagttg |
| 22981 | tagtactttc ttttgaactt ttaaatgcac cggccacggt |
| | ttgtggacca aaattatcca |
| 23041 | ctgaccttat taagaaccag tgtgtcaatt ttaattttaa |
| | tggactcact ggtactggtg |
| 23101 | tgttaactcc ttcttcaaag agatttcaac catttcaaca |
| | atttggccgt gatgtttctg |
| 23161 | atttcactga ttccgttcga gatcctaaaa catctgaaat |
| | attagacatt tcaccttgcg |
| 23221 | cttttggggg tgtaagtgta attacacctg gaacaaatgc |
| | ttcatctgaa gttgctgttc |
| 23281 | tatatcaaga tgttaactgc actgatgttt ctacagcaat |
| | tcatgcagat caactcacac |
| 23341 | cagcttggcg catatattct actggaaaca atgtattcca |
| | gactcaagca ggctgtctta |
| 23401 | taggagctga gcatgtcgac acttcttatg agtgcgacat |
| | tcctattgga gctggcattt |
| 23461 | gtgctagtta ccatacagtt tctttattac gtagtactag |
| | ccaaaaatct attgtggctt |
| 23521 | atactatgtc tttaggtgct gatagttcaa ttgcttactc |
| | taataacacc attgctatac |
| 23581 | ctactaactt ttcaattagc attactacag aagtaatgcc |
| | tgtttctatg gctaaaacct |
| 23641 | ccgtagattg taatatgtac atctgcggag attctactga |
| | atgtgctaat ttgcttctcc |
| 23701 | aatatggtag cttttgcaca caactaaatc gtgcactctc |
| | aggtattgct gctgaacagg |
| 23761 | atcgcaacac acgtgaagtg ttcgctcaag tcaaacaaat |
| | gtacaaaacc ccaactttga |
| 23821 | aatattttgg tggttttaat ttttcacaaa tattacctga |
| | ccctctaaag ccaactaaga |
| 23881 | ggtcttttat tgaggacttg ctctttaata aggtgacact |
| | cgctgatgct ggcttcatga |
| 23941 | agcaatatgg cgaatgccta ggtgatatta atgctagaga |
| | tctcatttgt gcgcagaagt |
| 24001 | tcaatggact tacagtgttg ccacctctgc tcactgatga |
| | tatgattgct gcctacactg |
| 24061 | ctgctctagt tagtggtact gccactgctg gatggacatt |
| | tggtgctggc gctgctcttc |
| 24121 | aaataccttt tgctatgcaa atggcatata ggttcaatgg |
| | cattggagtt acccaaaatg |
| 24181 | ttctctatga gaaccaaaaa caaatcgcca accaatttaa |
| | caaggcgatt agtcaaattc |
| 24241 | aagaatcact tacaacaaca tcaactgcat tgggcaagct |
| | gcaagacgtt gttaaccaga |
| 24301 | atgctcaagc attaaacaca cttgttaaac aacttagctc |
| | taattttggt gcaatttcaa |
| 24361 | gtgtgctaaa tgatatcctt tcgcgacttg ataaagtcga |
| | ggcggaggta caaattgaca |
| 24421 | ggttaattac aggcagactt caaagccttc aaacctatgt |
| | aacacaacaa ctaatcaggg |
| 24481 | ctgctgaaat cagggcttct gctaatcttg ctgctactaa |
| | aatgtctgag tgtgttcttg |
| 24541 | gacaatcaaa aagagttgac ttttgtggaa agggctacca |
| | ccttatgtcc ttcccacaag |
| 24601 | cagccccgca tggtgttgtc ttcctacatg tcacgtatgt |
| | gccatcccag gagaggaact |
| 24661 | tcaccacagc gccagcaatt tgtcatgaag gcaaagcata |
| | cttccctcgt gaaggtgttt |
| 24721 | ttgtgtttaa tggcacttct tggtttatta cacagaggaa |
| | cttcttttct ccacaaataa |
| 24781 | ttactacaga caatacattt gtctcaggaa attgtgatgt |
| | cgttattggc atcattaaca |
| 24841 | acacagttta tgatcctctg caacctgagc ttgactcatt |
| | caaagaagag ctggacaagt |
| 24901 | acttcaaaaa tcatacatca ccagatgttg atcttggcga |
| | catttcaggc attaacgctt |
| 24961 | ctgtcgtcaa cattcaaaaa gaaattgacc gcctcaatga |
| | ggtcgctaaa aatttaaatg |
| 25021 | aatcactcat tgaccttcaa gaattgggaa aatatgagca |
| | atatattaaa tggccttggt |
| 25081 | atgtttggct cggcttcatt gctggactaa ttgccatcgt |
| | catggttaca atcttgcttt |
| 25141 | gttgcatgac tagttgttgc agttgcctca agggtgcatg |
| | ctcttgtggt tcttgctgca |
| 25201 | agtttgatga ggatgactct gagccagttc tcaagggtgt |
| | caaattacat tacacataaa |
| 25261 | cgaacttatg gatttgttta tgagattttt tactcttaga |
| | tcaattactg cacagccagt |
| 25321 | aaaaattgac aatgcttctc ctgcaagtac tgttcatgct |
| | acagcaacga taccgctaca |
| 25381 | agcctcactc cctttcggat ggcttgttat tggcgttgca |
| | tttcttgctg tttttcagag |
| 25441 | cgctaccaaa ataattgcgc tcaataaaag atggcagcta |
| | gccctttata agggcttcca |
| 25501 | gttcatttgc aatttactgc tgctatttgt tacdatctat |
| | tcacatcttt tgcttgtcgc |
| 25561 | tgcaggtatg gaggcgcaat ttttgtacct ctatgccttg |
| | atatattttc tacaatgcat |
| 25621 | caacgcatgt agaattatta tgagatgttg gctttgttgg |
| | aagtgcaaat ccaagaaccc |
| 25681 | attactttat gatgccaact actttgtttg ctggcacaca |
| | cataactatg actactgtat |
| 25741 | accatataac agtgtcacag atacaattgt cgttactgaa |
| | ggtgacggca tttcaacacc |
| 25801 | aaaactcaaa gaagactacc aaattggtgg ttattctgag |
| | gataggcact caggtgttaa |
| 25861 | agactatgtc gttgtacatg gctatttcac cgaagtttac |
| | taccagcttg agtctacaca |
| 25921 | aattactaca gacactggta ttgaaaatgc tacattcttc |
| | atctttaaca agcttgttaa |
| 25981 | agacccaccg aatgtgcaaa tacacacaat cgacggctct |
| | tcaggagttg ctaatccagc |
| 26041 | aatggatcca atttatgatg agccgacgac gactactagc |
| | gtgcctttgt aagcacaaga |
| 26101 | aagtgagtac gaacttatgt actcattcgt ttcggaagaa |
| | acaggtacgt taatagttaa |
| 26161 | tagcgtactt ctttttcttg ctttcgtggt attcttgcta |
| | gtcacactag ccatccttac |
| 26221 | tgcgcttcga ttgtgtgcgt actgctgcaa tattgttaac |
| | gtgagtttag taaaaccaac |
| 26281 | ggtttacgtc tactcgcgtg ttaaaaatct gaactcttct |
| | gaaggagttc ctgatcttct |
| 26341 | ggtctaaacg aactaactat tattattatt ctgtttggaa |
| | ctttaacatt gcttatcatg |
| 26401 | gcagacaacg gtactattac cgttgaggag cttaaacaac |
| | tcctggaaca atggaaccta |
| 26461 | gtaataggtt tcctattcct agcctggatt atgttactac |
| | aatttgccta ttctaatcgg |
| 26521 | aacaggtttt tgtacataat aaagcttgtt ttcctctggc |
| | tcttgtggcc agtaacactt |
| 26581 | gcttgttttg tgcttgctgc tgtctacaga attaattggg |
| | tgactggcgg gattgcgatt |
| 26641 | gcaatggctt gtattgtagg cttgatgtgg cttagctact |
| | tcgttgcttc cttcaggctg |
| 26701 | tttgctcgta cccgctcaat gtggtcattc aacccagaaa |
| | caaacattct tctcaatgtg |
| 26761 | cctctccggg ggacaattgt gaccagaccg ctcatggaaa |
| | gtgaacttgt cattggtgct |
| 26821 | gtgatcattc gtggtcactt gcgaatggcc ggacactccc |
| | tagggcgctg tgacattaag |
| 26881 | gacctgccaa aagagatcac tgtggctaca tcacgaacgc |
| | tttcttatta caaattagga |
| 26941 | gcgtcgcagc gtgtaggcac tgattcaggt tttgctgcat |
| | acaaccgcta ccgtattgga |
| 27001 | aactataaat taaatacaga ccacgccggt agcaacgaca |
| | atattgcttt gctagtacag |
| 27061 | taagtgacaa cagatgtttc atcttgttga cttccaggtt |
| | acaatagcag agatattgat |
| 27121 | tatcattatg aggactttca ggattgctat ttggaatctt |
| | gacgttataa taagttcaat |
| 27181 | agtgagacaa ttatttaagc ctctaactaa gaagaattat |
| | tcggagttag atgatgaaga |
| 27241 | acctatggag ttagattatc cataaaacga acatgaaaat |
| | tattctcttc ctgacattga |
| 27301 | ttgtatttac atcttgcgag ctatatcact atcaggagtg |
| | tgttagaggt acgactgtac |
| 27361 | tactaaaaga accttgccca tcaggaacat acgagggcaa |
| | ttcaccattt caccctcttg |
| 27421 | ctgacaataa atttgcacta acttgcacta gcacacactt |
| | tgcttttgct tgtgctgacg |
| 27481 | gtactcgaca tacctatcag ctgcgtgcaa gatcagtttc |
| | accaaaactt ttcatcagac |
| 27541 | aagaggaggt tcaacaagag ctctactcgc cactttttct |
| | cattgttgct gctctagtat |
| 27601 | ttttaatact ttgcttcacc attaagagaa agacagaatg |
| | aatgagctca ctttaattga |
| 27661 | cttctatttg tgctttttag cctttctgct attccttgtt |
| | ttaataatgc ttattatatt |
| 27721 | ttggttttca ctcgaaatcc aggatctaga agaaccttgt |
| | accaaagtct aaacgaacat |
| 27781 | gaaacttctc attgttttga cttgtatttc tctatgcagt |
| | tgcatatgca ctgtagtaca |
| 27841 | gcgctgtgca tctaataaac ctcatgtgct tgaagatcct |
| | tgtaaggtac aacactaggg |
| 27901 | gtaatactta tagcactgct tggctttgtg ctctaggaaa |
| | ggttttacct tttcatagat |
| 27961 | ggcacactat ggttcaaaca tgcacaccta atgttactat |
| | caactgtcaa gatccagctg |
| 28021 | gtggtgcgct tatagctagg tgttggtacc ttcatgaagg |
| | tcaccaaact gctgcattta |
| 28081 | gagacgtact tgttgtttta aataaacgaa caaattaaaa |
| | tgtctgataa tggaccccaa |
| 28141 | tcaaaccaac gtagtgcccc ccgcattaca tttggtggac |
| | ccacagattc aactgacaat |
| 28201 | aaccagaatg gaggacgcaa tggggcaagg ccaaaacagc |
| | gccgacccca aggtttaccc |
| 28261 | aataatactg cgtcttggtt cacagctctc actcagcatg |
| | gcaaggagga acttagattc |
| 28321 | cctcgaggcc agggcgttcc aatcaacacc aatagtggtc |
| | cagatgacca aattggctac |
| 28381 | taccgaagag ctacccgacg agttcgtggt ggtgacggca |
| | aaatgaaaga gctcagcccc |
| 28441 | agatggtact tctattacct aggaactggc ccagaagctt |
| | cacttcccta cggcgctaac |
| 28501 | aaagaaggca tcgtatgggt tgcaactgag ggagccttga |
| | atacacccaa agaccacatt |
| 28561 | ggcacccgca atcctaataa caatgctgcc accgtgctac |
| | aacttcctca aggaacaaca |
| 28621 | ttgccaaaag gcttctacgc agagggaagc agaggcggca |
| | gtcaagcctc ttctcgctcc |
| 28681 | tcatcacgta gtcgcggtaa ttcaagaaat tcaactcctg |
| | gcagcagtag gggaaattct |
| 28741 | cctgctcgaa tggctagcgg aggtggtgaa actgccctcg |
| | cgctattgct gctagacaga |
| 28801 | ttgaaccagc ttgagagcaa agtttctggt aaaggccaac |
| | aacaacaagg ccaaactgtc |
| 28861 | actaagaaat ctgctgctga ggcatctaaa aagcctcgcc |
| | aaaaacgtac tgccacaaaa |
| 28921 | cagtacaacg tcactcaagc atttgggaga cgtggtccag |
| | aacaaaccca aggaaatttc |
| 28981 | ggggaccaag acctaatcag acaaggaact gattacaaac |
| | attggccgca aattgcacaa |
| 29041 | tttgctccaa gtgcctctgc attctttgga atgtcacgca |
| | ttggcatgga agtcacacct |
| 29101 | tcgggaacat ggctgactta tcatggagcc attaaattgg |
| | atgacaaaga tccacaattc |
| 29161 | aaagacaacg tcatactgct gaacaagcac attgacgcat |
| | acaaaacatt cccaccaaca |
| 29221 | gagcctaaaa aggacaaaaa gaaaaagact gatgaagctc |
| | agcctttgcc gcagagacaa |
| 29281 | aagaagcagc ccactgtgac tcttcttcct gcggctgaca |
| | tggatgattt ctccagacaa |
| 29341 | cttcaaaatt ccatgagtgg agcttctgct gattcaactc |
| | aggcataaac actcatgatg |
| 29401 | accacacaag gcagatgggc tatgtaaacg ttttcgcaat |
| | tccgtttacg atacatagtc |
| 29461 | tactcttgtg cagaatgaat tctcgtaact aaacagcaca |
| | agtaggttta gttaacttta |
| 29521 | atctcacata gcaatcttta atcaatgtgt aacattaggg |
| | aggacttgaa agagccacca |
| 29581 | cattttcatc gaggccacgc ggagtacgat cgagggtaca |
| | gtgaataatg ctagggagag |
| 29641 | ctgcctatat ggaagagccc taatgtgtaa aattaatttt |
| | agtagtgcta tccccatgtg |
| 29701 | attttaatag cttcttagga gaatgacaaa aaaaaaaaaa |
| | aaaaaaaaaa a |

While placement of the fluorescent protein within the coronavirus genome is preferred, additional preferred embodiments of the invention provide for the construction of virus-fluorescent fusion proteins that permit one of ordinary skill in the art to follow viral reproduction in an animal model. Either viral structural proteins or non-structural proteins can be used as fusion protein partners. Preferred structural proteins for use as fusion protein partners include but are not limited to a nucleocapsid phosphoprotein, a spike glycoprotein, a membrane glycoprotein, a small envelope protein, or a hemagglutinin-esterase glycoprotein. Sequences for each of these proteins have been disclosed in the art for a variety of coronaviruses, including the murine and SARS strains.

Model

The disclosed invention uses recombinant coronaviruses that are engineered to express a marker, such as a fluorescent protein. By infecting a model organism with the described recombinant coronavirus, one of ordinary skill in the art can use the recombinant virus to study the progression of viral replication in the host animal. Furthermore, the recombinant coronavirus model system has utility as an assay for identifying antiviral agents that slow or inhibit coronavirus replication.

The label used in the various aspects of the invention is a fluorescent protein. The native gene encoding the seminal protein in this class, green fluorescent protein (GFP) has been cloned from the bioluminescent jellyfish Aequorea victoria (Morin, J., et al., J. Cell Physiol (1972) 77:313-318). The availability of the gene has made it possible to use GFP as a marker for gene expression. The original GFP itself is a 283 amino acid protein with a molecular weight of 27 kD. It requires no additional proteins from its native source nor does it require substrates or cofactors available only in its native source in order to fluoresce. (Prasher, D. C., et al., Gene (1992) 111:229-233; Yang, F., et al., Nature Biotechnol (1996) 14:1252-1256; Cody, C. W., et al., Biochemistry (1993) 32:1212-1218.) Mutants of the original GFP gene have been found useful to enhance expression and to modify excitation and fluorescence, so that “GFP” in various colors, including reds and blues has been obtained. GFP-S65T (wherein serine at 65 is replaced with threonine) is particularly useful in the present invention method and has a single excitation peak at 490 nm. (Heim, R., et al., Nature (1995) 373:663-664); U.S. Pat. No. 5,625,048. Other mutants have also been disclosed by Delagrade, S., et al., Biotechnology (1995) 13:151-154; Cormack, B., et al., Gene (1996) 173:33-38 and Cramer, A., et al., Nature Biotechnol (1996) 14:315-319. Additional mutants are also disclosed in U.S. Pat. No. 5,625,048. By suitable modification, the spectrum of light emitted by the GFP can be altered. Thus, although the term “GFP” is often used in the present application, the proteins included within this definition are not necessarily green in appearance. Various forms of GFP exhibit colors other than green and these, too, are included within the definition of “GFP” and are useful in the methods and materials of the invention. In addition, it is noted that green fluorescent proteins falling within the definition of “GFP” herein have been isolated from other organisms, such as the sea pansy, Renilla reniformis. Any suitable and convenient form of GFP can be used to modify the infectious agents useful in the invention, both native and mutated forms.

In order to avoid confusion, the simple term “fluorescent protein” will be used; in general, this is understood to refer to the fluorescent proteins which are produced by various organisms, such as Renilla and Aequorea as well as modified forms of these native fluorescent proteins which may fluoresce in various visible colors, such as red, yellow, and cobalt, which are exhibited by red fluorescent protein (RFP), yellow fluorescent protein (YFP) or cobalt fluorescent protein (CFP), respectively. In general, the terms “fluorescent protein” and “GFP” or “RFP” are used interchangeably.

Because fluorescent proteins are available in a variety of colors, imaging with respect to more than a single color can be done simultaneously. For example, two different infective agents or three different infective agents each expressing a characteristic fluorescence can be administered to the organism and differential effects of proposed treatments evaluated. In addition, a single infectious organism could be labeled constitutively with a single color and a different color used to produce a fusion with a gene product either intracellular or that is secreted. Thus, the nucleotide sequence encoding a fluorescent protein having a color different from that used to label the organism per se can be inserted at a locus to be studied or as a fusion protein in a vector with a protein to be studied. Two-color imaging will be used to visualize targeting of the virus to particular sites in the model, such as the lungs. Further, one or more infective agents can each be labeled with a single color, a gene of interest with another color, and the host model tissue with a third color. For example, fluorescence-expressing coronavirus models will enable visualization of viral reproduction by whole body imaging.

The method of the disclosed invention can be used, to monitor the replication of the recombinant coronaviruses discussed above and the affect various antiviral agents such as chemotherapeutic agents and antiviral vaccines have on coronavirus reproduction.

The methods of the invention utilize infectious agents which have been modified to express the nucleotide sequence encoding a fluorescent protein, preferably of sufficient fluorescence intensity that the fluorescence can be seen in the subject without the necessity of any invasive technique. While whole body imaging is preferred because of the possibility of real-time observation, endoscopic techniques, for example, can also be employed or, if desired, tissues or organs excised for direct or histochemical observation.

The nucleotide sequence encoding the fluorescent protein may be introduced into the infectious agent by direct modification, such as modification of a viral genome to locate the fluorescent protein encoding sequence in a suitable position under the control sequences endogenous to the virus, or may be introduced into microbial systems using appropriate expression vectors.

The appropriately modified infectious agent is then administered to the subject in a manner which mimics, if desired, the route of infection believed used by the agent or by an arbitrary route. Administration may be by injection, gavage, oral, by aerosol into the respiratory system, by suppository, by contact with a mucosal surface in general, or by any suitable means known in the art to introduce infectious agents.

Although endoscopy can be used as well as excision of individual tissues, it is particularly convenient to visualize the migration of infective agent and infected cells in the intact animal through fluorescent imaging. This permits real-time observation and monitoring of progression of infection on a continuous basis, in particular, in model systems, in evaluation of potential anti-infective drugs and protocols. Thus, the inhibition of infection observed directly in test animals administered a candidate drug or protocol in comparison to controls which have not been administered the drug or protocol indicates the efficacy of the candidate and its potential as a treatment. In subjects being treated for infection, the availability of fluorescent imaging permits those devising treatment protocols to be informed on a continuous basis of the advisability of modifying or not modifying the protocol. In one embodiment, to screen for effective antiviral agents, recombinant coronaviruses that express fluorescently-labeled viral proteins are injected into a murine model to follow viral reproduction. Sites of viral infection are highly fluorescent and readily visualized by blue light excitation in a light box with a CCD camera and a GFP filter.

Suitable vertebrate subjects for use as models are preferably mammalian subjects, most preferably convenient laboratory animals such as rabbits, rats, mice, and the like. For closer analogy to human subjects, primates could also be used. Any appropriate vertebrate subject can be used, the choice being dictated mainly by convenience and similarity to the system of ultimate interest. Ultimately, the vertebrate subjects can be humans.

The following examples are offered to illustrate but not to limit the invention.

### EXAMPLE 1

A. Background

A dual-color fluorescence imaging model of tumor-host interaction based on an RFP-expressing tumor growing in GFP transgenic mice, enabling dual-color visualization of the tumor-stroma interaction including tumor angiogenesis and infiltration of lymphocytes in the tumor has been described. Transgenic mice expressing the GFP under the control of a chicken beta-actin promoter and cytomegalovirus enhancer were used as the host (Okabe, M., et al., FEBS Lett (1997) 407:315-319). All of the tissues from this transgenic line fluoresce green under blue excitation light. RFP-expressing B16F0 (B16F0-RFP) mouse melanoma cells were transduced with the pLNCX₂-DsRed-2-RFP plasmid. The B16F0-RFP tumor and GFP-expressing host cells could be clearly imaged simultaneously. High-resolution dual-color images enabled resolution of the tumor cells and the host tissues down to the single cell level. Host cells including fibroblasts, tumor infiltrating lymphocytes, dendritic cells, blood vessels and capillaries that express GFP, could be readily distinguished from the RFP-expressing tumor cells. This dual-color fluorescence imaging system should facilitate studies for understanding tumor-host interaction during tumor growth and tumor angiogenesis. The dual-colored chimeric system also provides a powerful tool to analyze and isolate tumor infiltrating lymphocytes and other host stromal cells interacting with the tumor for therapeutic and diagnostic/analytic purposes. The principles of this model are used in the dual-color imageable RFP-MHV-GFP-host infectious model of the invention.

B. Methods

Viruses and cells: The methods of de Haan, et al., Virol. (2002) 296:177-189 are followed. The MHV-A59 temperature-sensitive (ts) mutant LA16, the plaque-cloned MHV-JHM, and virus sample obtained after 19 undiluted passages of original plaque-cloned MHV-JHM (JHM19th) are employed. Mouse DBT cells are used for RNA transfection and propagation of viruses.

The methods of Kim, K. H., J. Virol. (1995) 69:2313-2321 are followed, in the following sections:

Preparation of virus-specific intracellular RNA and Northern (RNA) blotting: Virus-specific RNAs are extracted from virus-infected cells. 1.5 mg of intracellular RNA is denatured and electrophoresed through a 1% agarose gel containing formaldehyde. The separated RNA was blotted onto nylon filters. The RNA on the filters is hybridized with ³²P-labeled probes specific for the various regions of MHV RNA.

RNA transcription and transfection: Plasmids are linearized by XbaI digestion and transcribed in vitro with T7 RNA polymerase. Lipofection is used for RNA transfection.

Isolation of clones containing the DIssA-specific sequence: For the amplification of a DIssA-related subgenomic RNA, cDNA is first synthesized from intracellular RNA, using as a primer oligonucleotide 1116 (5′-CTGAAACTCTTTTCCCT-3′)(SEQ ID NO: XX), which binds to positive-strand MHV mRNA 7 at nucleotides 250 to 267 from the 5′ end of mRNA 7. MHV-specific cDNA is then incubated with oligonucleotide 78 (5′-AGCTTTACGTACCCTCTCTACTATAAAACTCTTGTAGTTT-3′)(SEQ ID NO: XX), which binds to antileader sequence of MHV RNA, in PCR buffer (0.05 M KCl, 0.01 M Tris hydrochloride [pH 8.3], 0.0025 M MgCl2, 0.01% gelatin, 0.17 mM of each deoxynucleoside triphosphate, 5 U of Taq polymerase [Promega]) at 93.8° C. for 30 s, 37.8° C. for 45 s, and 72.8° C. for 100 s for 25 cycles DIssA subgenomic RNA were separated by agarose gelelectrophoresis and hybridized with a probe which corresponds to 1.5 to 1.7 kb from the 3′ end of MHV genomic RNA. This probe hybridizes with all MHV mRNAs. The DIssA subgenomic RNA-specific RT-PCR product is eluted from the gel and cloned into the TA cloning vector (Invitrogen). Clones containing DIssA-specific sequence are isolated by colony hybridization using the probe that was used for Southern blot analysis. For amplification of DIssA RNA, cDNA is first synthesized from gel-purified DIssA RNA by using oligonucleotide 1116 as a primer. DIssA-specific cDNA is then incubated with oligonucleotide 10121 (5′-GAAGGGTTGTATGTGTTG-3′)(SEQ ID NO: XX), which binds to negative strand MHV RNA at nucleotides 798 to 815 from the 5′ end of gene 2, in PCR buffer under the PCR conditions described above. The DIssA-specific RT-PCR product is eluted from the preparative gel and cloned into the TA cloning vector. Clones containing DIssA-specific sequences are isolated by colony hybridization using the probe which hybridizes at MHV gene 2-1.

Construction of Mouse Hepatitis Full-Length cDNA linked to RFP: DIssA is a naturally occurring self-replicating DI RNA with nearly intact genes 1 and 7 of the MHV as noted above. We will flank gene 1 and gene 7 of the cDNA of MHV, plus the RFP gene in bacterial artificial chromosome (BAC) pBeloBACII, at its 5′ end by the CMV immediate-early promoter and at its 3′ end followed by poly(A) tail in turn followed by the hepatitis delta virus ribozyme and the bovine GH termination and polyadenylation sequences pBAC-MHV-RFP (see, Almazan, F., et al, PNAS (2000) 97:5516-5521).

Transfection and Recovery of an Infectious Virus from a cDNA Clone: The methods of Almazan, F., et al., PNAS (2000) 97:5516-5521 are used in this procedure. The mouse DBT cells are used for transfected by pBAC-MHV-RFP. After an incubation period of 2 days, the cell supernatant was harvested and passaged six times on fresh DBT cells. Virus present in the cell supernatant was analyzed by plaque tritation and RT-PCR.

RFP Expression Vectors (See, Yang, M., Proc. Natl. Acad. Sci. USA (2002) 99:3824-3829). The pLNCX₂ vectors is purchased from CLONTECH Laboratories, Inc. (Palo Alto, Cailf.). The pLNCX₂ vector contains the neomycin resistance gene for antibiotic selection in eukaryotic cells. The red fluorescent protein (RFP), (DsRed2, CLONTECH Laboratories, Inc., Palo Alto, Cailf.), is inserted in the pLNCX₂ vector at the Egl II and Not I sites.

RFP vector production (See, Yang, M., Proc. Natl. Acad. Sci. USA (2002) 99:3824-3829). For retroviral transduction, PT67, an NIH3T3-derived packaging cell line, expressing the 10 Al viral envelope, is purchased from CLONTECH Laboratories, Inc. PT67 cells are cultured in DME (Irvine Scientific, Santa Ana, Calif.) supplemented with 10% heat-inactivated fetal bovine serum (FBS) (Gemini Bio-products, Calabasas, Calif.). For vector production, packaging cells (PT67), at 70% confluence, are incubated with a precipitated mixture of DOTAP™ reagent (Boehringer Mannheim), and saturating amounts of pLEIN-GFP or pLNCX₂-DsRed-2-RFP plasmid for 18 hours. Fresh medium is replenished at this time. The cells are examined by fluorescence microscopy 48 hours post-transfection. For selection, the cells are cultured in the presence of 500 μg/ml- 2000 μg/ml of G418 increased in a step-wise manner (Life Technologies, Grand Island, N.Y.) for seven days.

Dual-color imaging of virus-host interaction: After infection of recombinant coronavirus to the GFP transgenic mice, the fresh tissues are cut into ˜1 mm³ pieces. The tissues are digested with trypsin/EDTA at 37 C.° for 10 minutes before examination. After trypsinization, tissues are put on precleaned microscope slides (Fisher Scientific, Pittsburgh, Pa.) and covered with a cover slip (Fisher Scientific). The tissues are pressed to become thin enough by pushing the cover slip to display the intact vasculature on the slides. The GFP-fluorescing host cells that are infected with the coronavirus can be readily observed under fluorescence microscopy. Laser-based systems will be used for whole-body dual-color imaging of the chimeric system (please see below). All fluorescence results will be confirmed by standard immunohistochemical techniques to identify host all types infected by the RFP-MHV.

Fluorescence imaging (See, Yang, M., Proc. Natl. Acad. Sci. USA (2002) 99:3824-3829). A Leica fluorescence stereo microscope model LZ12 equipped with a mercury 50W lamp power supply is used for initial lower resolution imaging. For visualization of both GFP and RFP fluorescence simultaneously, excitation is produced through a D425/60 band pass filter and 470 DCXR dichroic mirror. Emitted fluorescence is collected through a long pass filter GG475 (Chroma Technology, Brattleboro, Vt.). Macroimaging is carried out in a light box (Lightools Research, Encinitas, Calif.). Fluorescence excitation of both GFP and RFP tumors is produced in the lightbox through an interference filter (440+/−20 nm) using slit fiber optics. Fluorescence is observed through a 520 nm long pass filter. Images from the microscope and light box are captured on a Hamamatsu C5810 3-chip cool color CCR camera (Hamamatsu Photonics Systems, Bridgewater, N.J.). Laser-based imaging is carried out with the Spectra Physics model 3941-M1BB dual photon laser, Photon Technology Intl. model GL-3300 nitrogen laser and the Photon Technology Intl. model GL-302 dye laser. Images are processed for contrast and brightness and analyzed with the use of Image Pro Plus 4.0 software (Media Cybernetics, Silver Springs, Md.). High resolution images of 1024×724 pixels are captured directly on an IBM PC or continuously through video output on a high resolution Sony VCR model SLV-R1000 (Sony Corp., Tokyo Japan).

Multiphoton confocal microscopy (Wang, W., et al., Cancer Research (2002) 6278-6288). The dual photon laser (Spectra-Physics model 3941-M1BB) is also used with the Radiance 2000 multiphoton system (Bio-Rad, Hercules, Calif.) at 960 nm, the optimal wavelength for GFP fluorescence. The images are collected using Bio-Rad's Lasersharp 2000 software. Excitation is confmed only to the optical section being observed. No excitation of the fluorophore will occur at 960 run wavelength not in the plane of focus. The Millenia, Tsunami Ti:Sapphire laser, an accessory for the Spectra Physics model 3941-M1BB dual photon laser, has long wavelength optics (beyond 1,000 nm) for RFP multiphoton imaging. Images are processed with Image Pro Plus 4.0 software.

Spectral resolution. Spectral imaging, is the generation of images containing a high-resolution optical spectrum at every pixel, to “unmix” the viral RFP signal from that of the GFP-labeled host. The standard GFP-mouse imaging system (long-pass emission filter) is modified by replacing the usual color camera with the cooled monochrome camera (Roper Scientific CCD thermo-cooled digital camera) and a liquid crystal tunable filter (CRI, Inc., Woburn, Mass.) positioned in front of a conventional macro-lens. Typically, a series of images is taken every 10 nm from 500 to 650 nm and assembled automatically in memory into a spectral “stack.” Using pre-defined GFP or RFP and autofluorescence spectra, the image can be resolved into different images using a linear combination chemometrics-based algorithm that generates images containing only the autofluorescence signals or only the GFP or RFP signals, now visible against essentially a black background. Using spectral autofluorescence subtraction, sensitivity is enhanced due to improvements in signal to noise ratio. The advantages provided by the GFP- or RFP-labeled tumor models, which allow noninvasive, and highly selective imaging, are further enhanced by using wavelength-selective imaging techniques and analysis to image tumors on deep organs such as the lung (personal communication, Richard Levenson, CRI, Inc., Woburn, Mass.).

Depth of imaging: External visualization of single cells or microscopic colonies of viral infected cells on internal organs is one goal of this application. Imaging of this power requires reducing scatter of excitation and emission light. Multiphoton and single photon lasers will be used for deeper penetration in the living animal. Confocal microscopy will also be used in conjunction with the multiphoton laser. The relatively high wave length of the excitation light, about 470 nm (960 nm for GFP dual photon and about 1,220 nm for RFP dual photon), will not damage tissue. The multiphoton confocal system will highly limit the irradiation area further protecting the host tissues. Skin-flaps also greatly reduce scatter which we have already shown to enable external single-cell imaging. Use of the long wave length Ds-Red-2-RFP also reduces scatter.

C. Results

The infected mice are treated with various drug regimens and evaluated for replication of the virus with and without the presence of the drug. Drugs that succeed in reducing viral replication are identified as successful candidates as therapeutic agents.

Similarly, mice subjected to immunization procedures to be tested are challenged after immunization with infectious levels of MHV coronavirus. The ability of the subject to resist infection after exposure is then evaluated.

1. A labeled coronavirus protein or fragment thereof coupled to a fluorescent protein.

2. The labeled coronavirus protein of claim 1, wherein the protein is a structural protein or a non-structural protein.

3. The labeled coronavirus protein of claim 2, wherein the structural protein is selected from the group consisting of a nucleocapsid phosphoprotein, spike glycoprotein, a membrane glycoprotein, a small envelope protein, or a hemagglutinin-esterase glycoprotein.

4. The labeled coronavirus protein of claim 2, wherein the structural protein is a SARS spike glycoprotein (SEQ ID NO:5).

5. The labeled coronavirus protein of claim 2, wherein the structural protein is a SARS small envelope protein (SEQ ID NO:6).

6. The labeled coronavirus protein of claim 2, wherein the structural protein is a SARS membrane glycoprotein (SEQ ID NO:7).

7. The labeled coronavirus protein of claim 1 wherein the fluorescent protein is a green or red protein.

8. An imageable animal model of infection comprising a coronavirus encoding the labeled coronavirus protein of claim 1.

9. The imageable animal model of claim 8 that is a fluorescent protein-expressing host.

10. The imageable animal model of claim 9, wherein the animal model comprises a transgenic green fluorescent protein-expressing mouse.

11. A method to screen antiviral drugs, comprising:
providing a test group of animals and a control group of animals, wherein the animals of each group comprise the animal model of claim 8;
administering to the test group an antiviral drug candidate;
monitoring fluorescence emissions produced by the test group and the control group;
comparing the fluorescence emissions produced by the test group to the control group; and
selecting the antiviral drug candidate that reduces fluorescence in the test group relative to the control group.

12. A method to screen effective antiviral vaccines, comprising:
providing a test group of animals and a control group of animals, wherein the animals of each group comprise the animal model of claim 8;
administering to the test group an antiviral vaccine candidate;
monitoring fluorescence emissions produced by the test group and the control group;
comparing the fluorescence emissions produced by the test group to the control group; and
selecting the antiviral vaccine candidate that reduces fluorescence in the test group relative to the control group.


SEQUENCE LISTING (7 sequences)

SEQ ID NO: 1 - DNA, 29751 bp, Coronavirus
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttagatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaa

SEQ ID NO: 2 - DNA, 17 bp, Artificial Sequence
  Primer
ctgaaactcttttccct

SEQ ID NO: 3 - DNA, 40 bp, Artificial Sequence
  Primer
agctttacgtaccctctctactataaaactcttgtagttt

SEQ ID NO: 4 - DNA, 18 bp, Artificial Sequence
  Primer
gaagggttgtatgtgttg

SEQ ID NO: 5 - DNA, 3768 bp, Coronavirus
  misc_feature (1)...(3768): derived from 21492...25259 of SEQ ID NO1
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 6 - DNA, 231 bp, Coronavirus
  misc_feature (1)...(231): derived from 26117...26347 of SEQ ID NO1
atgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaa

SEQ ID NO: 7 - DNA, 666 bp, Coronavirus
  misc_feature (1)...(666): derived from 26398...27063 of SEQ ID NO1
atggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaa